(19) United States
(12) Patent Application Publication
(10) Pub. No.: US 2006/0041963 A1
(43) Pub. Date: Feb. 23, 2006

(54) Double-type Kalanchoe interspecific hybrids

(75) Inventors: Knud Jepsen, Hinnerup (DK); Ellen Christensen, Hinnerup (DK)

Correspondence Address: FOLEY AND LARDNER LLP;SUITE 500, 3000 K STREET NW, WASHINGTON, DC, 20007, US

(73) Assignee: Knud Jepsen a/s

(21) Appl. No.: 11/011,618

(22) Filed: Dec. 15, 2004

(30) Foreign Application Priority Data
Aug. 18, 2004 (QZ) 2004/1546
Aug. 18, 2004 (QZ) 2004/1547
Aug. 18, 2004 (QZ) 2004/1548

(51) Int. Cl. A01H 5/00

(52) U.S. Cl. 800/323

(57) ABSTRACT

New, distinct and stable cultivars of double-flowering Kalanchoe interspecific hybrids are disclosed. Double-flowering Kalanchoe interspecific hybrids produce one or more flowers with at least 5 full or partial petals per flower. Double-type K. aromatica×K. blossfeldiana, K. grandiflora×K. blossfeldiana, K. laciniata×K. blossfeldiana, K. pumila×K. blossfeldiana, K. marmorata×K. blossfeldiana, K. porphyrocalux×K. blossfeldiana, K. jongmansii×K. blossfeldiana, K. pinnata×K. blossfeldiana and K. daigremontiana×K. blossfeldiana are disclosed. The double-type flowering characteristic has been combined with many desirable Kalanchoe traits including different flower colors, growth characteristics and plant habit. Methods for the reliable breeding of the double-type characteristic into diverse Kalanchoe genetic backgrounds, as well as methods for increasing the number of petals per flower, are disclosed.

### CROSS-REFERENCE TO RELATED PATENT APPLICATIONS

This is a continuation of European Community Variety Rights application serial Nos. 2004/1546, 2004/1547, and 2004/1548, all filed on Aug. 18, 2004, respectively.

### FIELD OF INVENTION

The present invention relates to new, distinct, and stable cultivars of double-type Kalanchoe interspecific hybrids that produce one or more flowers with at least 5 full or partial petals per flower. The double-type Kalanchoe interspecific hybrids of the present invention include Kalanchoe aromatica×K. blossfeldiana, K. grandiflora×K. blossfeldiana, K. laciniata×K. blossfeldiana, K. pumila×K. blossfeldiana, K. marmorata×K. blossfeldiana, K. porphyrocalux×K. blossfeldiana, K. jongmansii×K. blossfeldiana, K. pinnata×K. blossfeldiana, and K. daigremontiana×K. blossfeldiana. The double-type interspecific hybrid plants produce one or more flowers having at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more than 40 petals per flower. The double-type characteristic has been successfully introgressed from double-type K. blossfeldiana into other single-type (4 petals per flower) Kalanchoe species. The double-type interspecific hybrid plants can be used as either male or female parents in crosses to the parents, other selections from the same species as the parents, or selections from other Kalanchoe species. The double-type interspecific hybrid plants can also be selfed. The double-type flowering characteristic from K. blossfeldiana has been combined with many desirable traits from other Kalanchoe species including different flower colors, superior growth characteristics and improved plant habit. Methods for the reliable breeding of the double-type characteristic from K. blossfeldiana, or Kalanchoe interspecific hybrid plants, into diverse single-type Kalanchoe genetic backgrounds, as well as methods for increasing the number of petal per flower, are disclosed.

### BACKGROUND OF THE INVENTION

The present invention relates generally to the field of ornamental Kalanchoe plants. The genus of Kalanchoe belongs to the sedum family (Crassulaceae). There are more than 100 different species of Kalanchoe of which more than 60 are found in Madagascar. Many species are found in South Africa and a few in Asia and South America. Kalanchoe belongs to the succulent plant group, which are characterized by their turgid leaves. The leaves enable members of this genus to survive drought conditions. Consequently, Kalanchoe are useful ornamental plants because they can survive in less than optimal growing conditions.

Species of Kalanchoe are described in J. T. Baldwin, Amer. J. Bot. 25: 572-579 (1938). Each species of Kalanchoe has traits a breeder would like to incorporate into commercial cultivars. Other traits a breeder will try to avoid.

K. blossfeldiana is a valuable ornamental species because it can be readily grown from seed, or reproduced asexually from cuttings, and produces abundant flowers that remain fresh for several weeks. Generally, K. blossfeldiana plants are highly branched and under certain conditions the height of the branches may be up to 30-45 cm. K. blossfeldiana plants generally produce erect flowers, oblong or ovate-oblong leaves, with a corolla that is red.

K. blossfeldiana can be crossed with numerous other Kalanchoe species to combine advantageous characteristics into unique new cultivars. Among the numerous interspecific hybrids that may be created are K. aromatica×K. blossfeldiana, K. grandiflora×K. blossfeldiana, K. laciniata×K. blossfeldiana, K. pumila×K. blossfeldiana, K. marmorata×K. blossfeldiana, K. porphyrocalux×K. blossfeldiana, K. jongmansii×K. blossfeldiana, K. pinnata×K. blossfeldiana, and K. daigremontiana×K. blossfeldiana.

The breeding of Kalanchoe interspecific hybrids with double-type, rose-like flowers, with more than 5 petals per flower, offers a unique flower form that can be combined with diverse Kalanchoe plant characteristics from different species, thereby expanding the range of phenotypic characteristics available in this popular horticultural plant.

### SUMMARY OF THE INVENTION

An object of the present invention is to provide a Kalanchoe interspecific hybrid plant having one or more double-type flowers with at least 5 full or partial petals per flower.

Another object of the present invention is to provide double-type Kalanchoe interspecific hybrid plants including K. aromatica×K. blossfeldiana, K. grandiflora×K. blossfeldiana, K. laciniata×K. blossfeldiana, K. pumila×K. blossfeldiana, K. marmorata×K. blossfeldiana, K. porphyrocalux×K. blossfeldiana, K. jongmansii×K. blossfeldiana, K. pinnata×K. blossfeldiana, and K. daigremontiana×K. blossfeldiana interspecific hybrids.

Yet another object of the present invention is to provide a Kalanchoe interspecific hybrid plant in which substantially all the flowers produced by the plant are double-type with at least 5 full or partial petal per flower.

Another object of the present invention is to provide a Kalanchoe interspecific hybrid plant wherein a double-type flower has at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more than 40 petals per flower.

An object of the present invention is to provide a method for breeding a double-type Kalanchoe interspecific hybrid plant that produces one or more flowers with at least 5 full or partial petals per flower comprising the steps of (a) crossing a double-type K. blossfeldiana double-type plant, either as the male or female parent, with a single-type Kalanchoe plant selected from the group consisting of K. aromatica, K. grandiflora, K. laciniata, K. pumila, K. marmorata, K. porphyrocalux, K. jongmansii, K. pinnata and K. daigremontiana; and (b) selecting progeny that produce one or more flowers with at least 5 full or partial petals per flower.

Another object of the present invention is to provide a method for breeding a double-type Kalanchoe interspecific hybrid plant that produces one or more flowers with at least 5 full or partial petals per flower wherein the double-type interspecific progeny plant is back-crossed with one of the parents.

Another object of the present invention is provide a method for breeding a double-type Kalanchoe interspecific hybrid plant that produces one or more flowers with at least 5 full or partial petals per flower comprising the step of selfing a double-type Kalanchoe interspecific hybrid selected from the group consisting of K. aromatica×K. blossfeldiana, K. grandiflora×K. blossfeldiana, K. laciniata×K. blossfeldiana, K. pumila×K. blossfeldiana, K. marmorata×K. blossfeldiana, K. porphyrocalux×K. blossfeldiana, K. jongmansii×K. blossfeldiana, K. pinnata×K. blossfeldiana and K. daigremontiana×K. blossfeldiana.

Yet another object of the present invention is to provide a method for breeding a double-type Kalanchoe interspecific hybrid plant that produces one or more flowers with at least 5 full or partial petals per flower comprising the steps of (a) crossing a double-type Kalanchoe interspecific hybrid selected from the group consisting of K. aromatica×K. blossfeldiana, K. grandiflora×K. blossfeldiana, K. laciniata×K. blossfeldiana, K. pumila×K. blossfeldiana, K. marmorata×K. blossfeldiana, K. porphyrocalux×K. blossfeldiana, K. jongmansii×K. blossfeldiana, K. pinnata×K. blossfeldiana and K. daigremontiana×K. blossfeldiana, as the male or female parent, with single or double-type Kalanchoe plant of the same or different species; and (b) selecting a progeny plant that produces double-type flowers.

### BRIEF DESCRIPTION OF THE DRAWINGS

The patent or application file contains at least one drawing executed in color. Copies of this patent or patent application publication with color drawings will be provided by the Office upon request and payment of the necessary fees.

FIG. 1. A top perspective view of a typical potted flowering plant of Kalanchoe cultivar ‘KJ 2003 0761’ 18 weeks after planting of cutting.

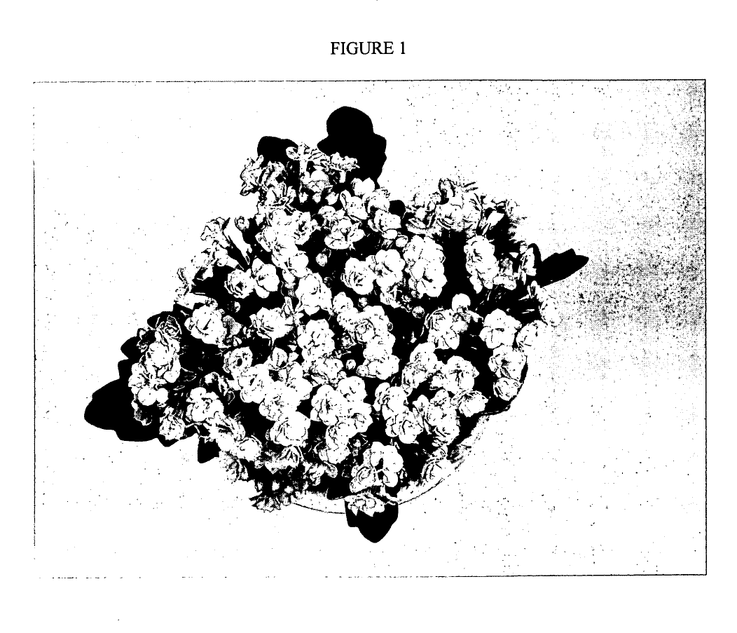

FIG. 2. A side perspective view of a typical potted flowering plant of Kalanchoe cultivar ‘KJ 2003 0761’ 18 weeks after planting of cutting.

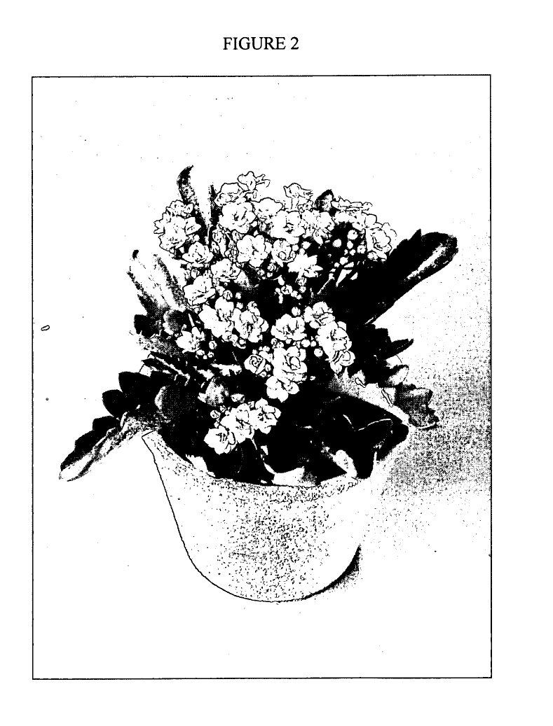

FIG. 3. Representative plant parts of Kalanchoe cultivar ‘KJ 2003 0761’: A. Inflorescence; B. Inside a flower; C. Flower bud, site; D. Flower; E. Flower bud, top; F. Petal; G. Pistil; H. Sepal; I. Mature leaf; J. Young leaf.

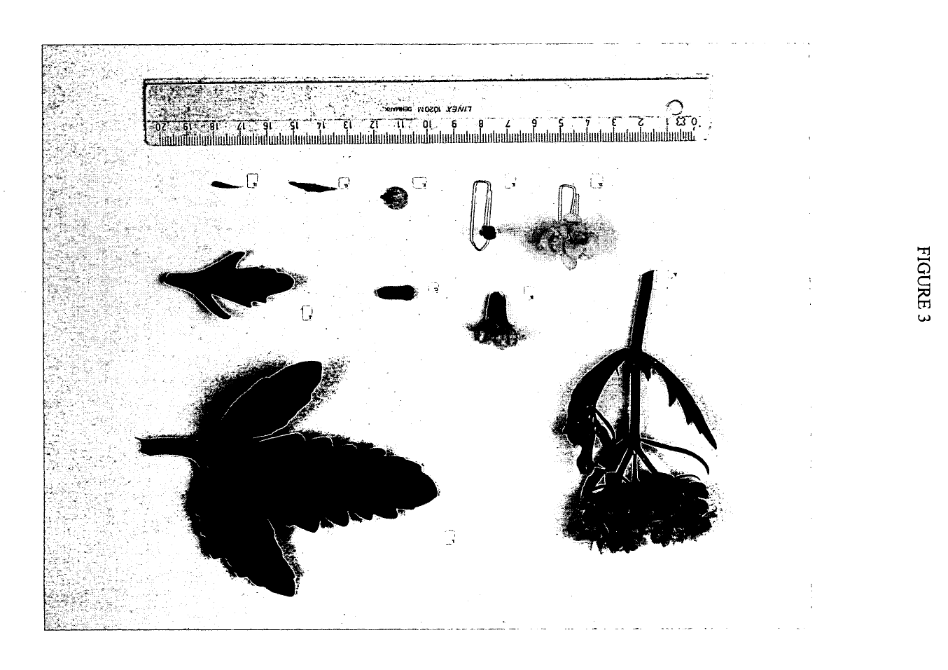

FIG. 4. A top perspective view of a typical potted flowering plant of Kalanchoe cultivar ‘KJ 2003 0638’ 18 weeks after planting of cutting.

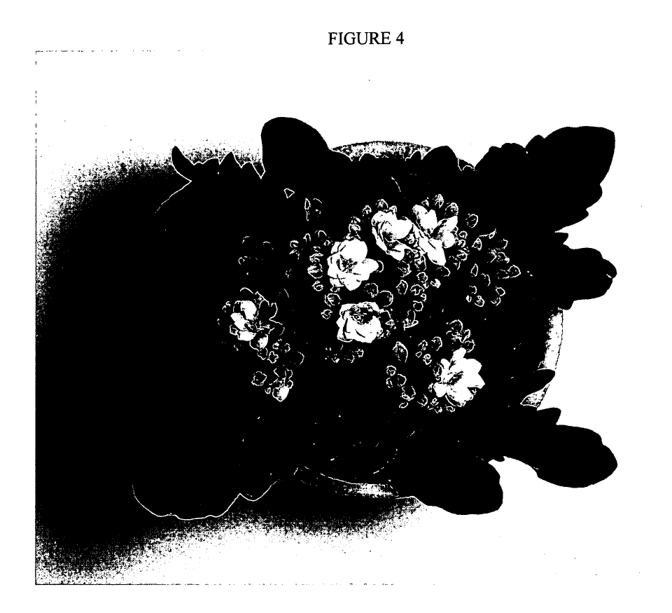

FIG. 5. A side perspective view of a typical potted flowering plant of Kalanchoe cultivar ‘KJ 2003 0638’ 18 weeks after planting of cutting.

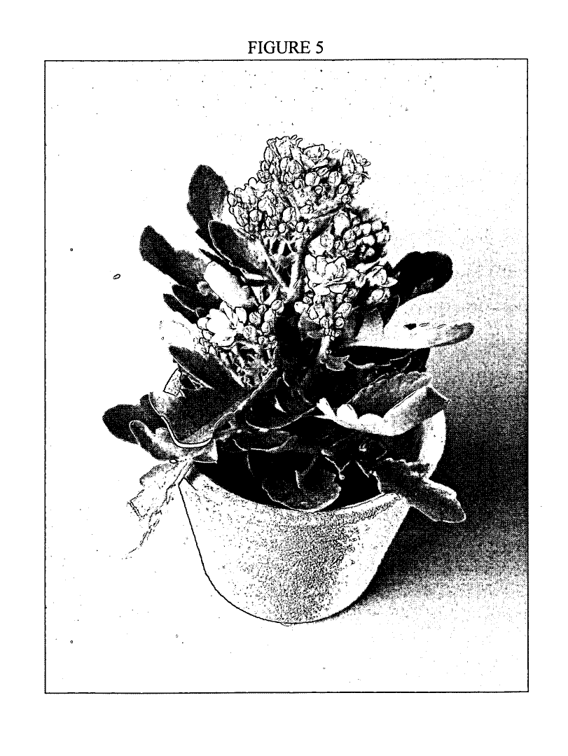

FIG. 6. Representative plant parts of Kalanchoe cultivar ‘KJ 2003 0638’: A Inflorescence; B. Flower, just opened; C. Flower opened one week, D. Flower bud, top; E. Inside a flower; F. Flower bud, site; G. Petal; H. Pistil ; I. Sepal; J. Mature leaf; and K. Young leaf.

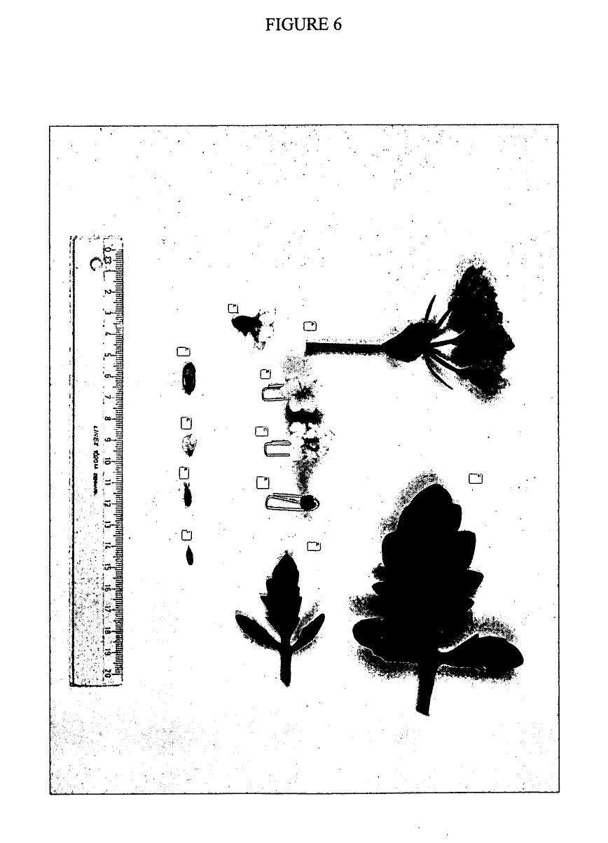

FIG. 7. A top perspective view of a typical potted flowering plant of Kalanchoe cultivar ‘KJ 2003 0747’ 16 weeks after planting of cutting.

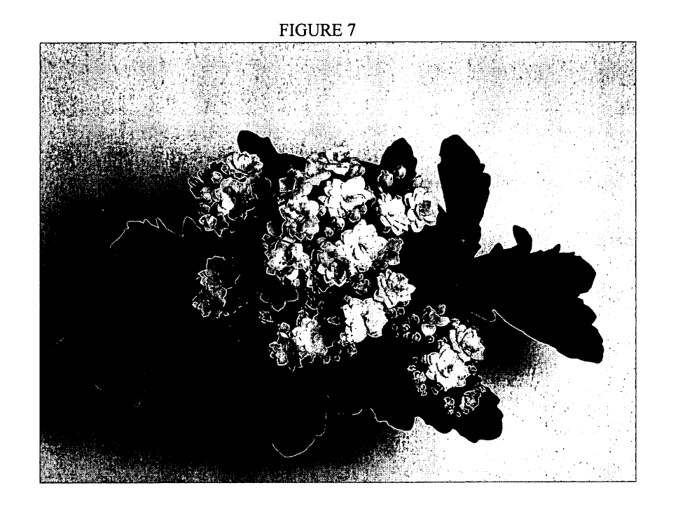

FIG. 8. A side perspective view of a typical potted flowering plant of Kalanchoe cultivar ‘KJ 2003 0747’ 16 weeks after planting of cutting.

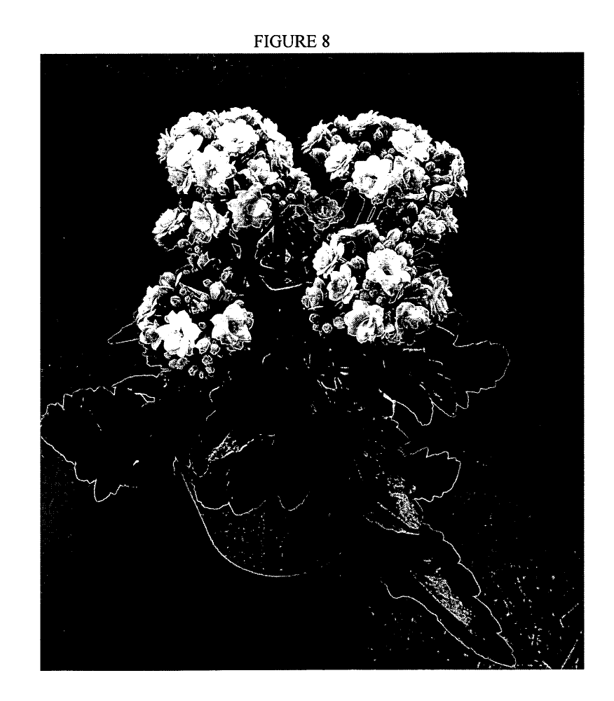

FIG. 9. Representative plant parts of Kalanchoe cultivar ‘KJ 2003 0747’: A. Inflorescence; B. Flower, just opened; C. Flower, opened one week; D. Color of a faded flower; E. Flower bud from the top; F. Inside a flower; G. Flower bud from the site; H. Petal; I. Sepal; J. Pistil; K. Mature leaf and L. Young leaf.

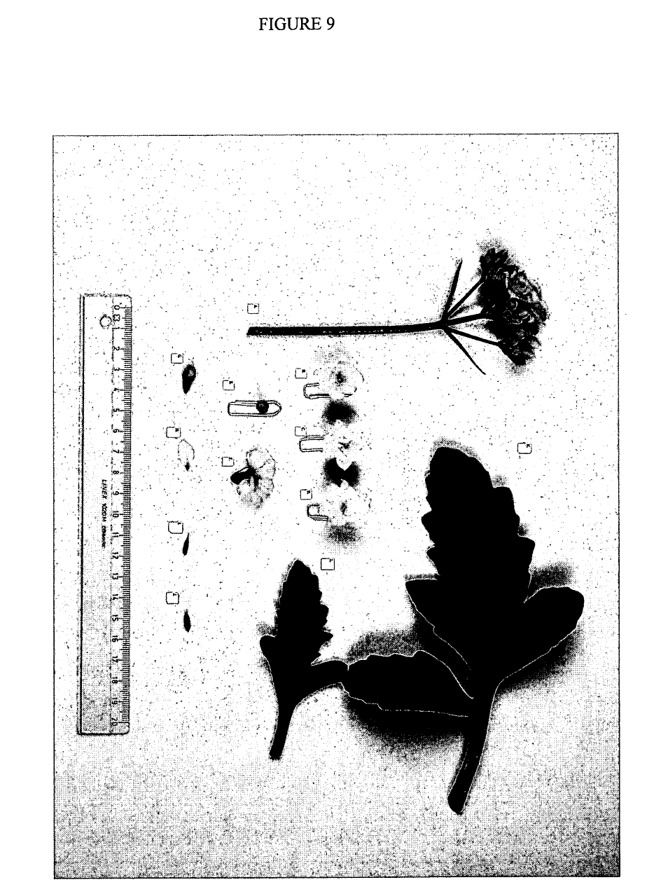

### DETAILED DESCRIPTION

As used herein, “single”, or “single-flowering”, or “single-type” are each defined as the typical Kalanchoe plant which produces flowers having 4 petals per flower.

As used herein, “double”, “double-flowering”, or “double-type” are each defined as a Kalanchoe interspecific hybrid plant which produces one or more flowers having at least 5 full or partial petals per flower. Preferably, the double-type Kalanchoe interspecific hybrid plant of the instant invention has substantially all double-type flowers. The double-type flowers of the instant invention have about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more than 40 petals per flower. A double-type Kalanchoe interspecific hybrid plant of the instant invention may produce flowers with a relatively uniform number of petals per flower and this characteristic is stable through asexual reproduction. Alternatively, a double-type Kalanchoe interspecific hybrid plant of the instant invention may produce flowers with a wide range in the number of petals per flower and this characteristic is also stable through asexual reproduction.

As used herein, the “degree of doubleness per flower” is defined as a measure of the number of extra full or partial petals per flower produced beyond the number 4 normally found on Kalanchoe single-type plant. The greater the degree of doubleness per flower, the greater the number of full or partial petals produced per flower.

As used herein “the degree of doubleness per plant” is defined as a measure of the number of flowers per plant which have at least 5 petals per flower. The greater the degree of doubleness per plant, the higher is the percentage of total flowers produced by the plant which have at least 5 full or partial petals per flower.

As used herein, “interspecific hybrid” includes the progeny from the cross of two different species of Kalanchoe, as well as progeny resulting from subsequent backcrossing to one of the parents. This backcrossing to one of the parents may be conducted one or more times with the goal of stably combining the double-type trait with desired characteristics.

K. blossfeldiana can be crossed with numerous other Kalanchoe species to combine advantageous characteristics into unique new cultivars. Among the numerous interspecific hybrids that may be created are K. aromatica×K. blossfeldiana, K. grandiflora×K. blossfeldiana, K. laciniata×K. blossfeldiana, K. pumila×K. blossfeldiana, K. marmorata×K. blossfeldiana, K. porphyrocalux×K. blossfeldiana, K. jongmansii×K. blossfeldiana, K. pinnata×K. blossfeldiana, and K. daigremontiana×K. blossfeldiana.

Advantageous traits obtained from K. aromatica plants include: 1) excellent petal shape, 2) good foliage, 3) small size of foliage, 4) good branching habit, 5) fast growth habit, 6) good rooting habit, 7) numerous branched roots and 8) production of many cuttings. Traits from K. aromatica that a breeder generally attempts to avoid include: 1) small flower size, 2) petals bending back-wards 3) soft hanging stem, 4) hairy stem texture and 5) poor post-production longevity.

Advantageous traits obtained from K. grandiflora plants include: 1) yellow coloration of flower, 2) fragrance of flower, 3) rounded shape of leaf, 4) medium-sized leaf, 4) red coloration on the stem, 5) same red coloration on the margin of the leaves and 6) vigorous rooting habit. Traits from K. grandiflora that a breeder will attempt to avoid include: 1) too strongly built plant, 2) crispy leaf texture that breaks easily, 3) grayish leaf color, and 4) poor post-production longevity.

K. pumila plants may include such advantageous traits as: 1) pink-purple coloration of flower, 2) attractive petal shape; 3) small leaves, 4) pink coloration on the stem and 5) vigorous rooting habit. K. pumila plants may have traits that should be avoided in a breeding program include: 1) petals bending backward, 2) soft hanging stem, 3) grayish leaf color, 4) poor fixation of the leaf to the stem, 5) long petiole with a small diameter, 6) poor branching habit and 7) poor post-production longevity.

K. marmorata plants may include such advantageous traits as: 1) white coloration of flower, 2) large size of flower 3) good fixation of leaf to the stem, 4) strong upright growth and 5) vigorous rooting habit. K. marmorata plants may have traits that should be avoided in the breeding program include: 1) crispy leaf texture that breaks easily and 2) dreary green with yellow spots coloration of foliage.

Advantageous traits obtained from K. porphyrocalux plants include: 1) bell-shaped flower, 2) dark purple coloration of petal, 3) smooth texture of petal, 4) green leaf color, 5) small, harmonic, upright and compact growth habit, 6) good fixation of leaf to the stem and 7) production of many cuttings. Traits from K. porphyrocalux that should be avoided in breeding programs include: 1) poor branching habit and 2) weak growth habit.

Advantageous traits obtained from K. jongmansii plants include: 1) yellow coloration of flower, 2) upright, bell-shaped flower, 3) small size of foliage, 4) strong stem, 5) vigorous rooting habit and 6) production of many cuttings. Traits from K. jongmansii that should be avoided in breeding programs include: 1) open flower shape, 2) vigorous and delicate plant growth habit, 3) small size of foliage, 4) small creeping growth habit and 5) poor post-production longevity.

K. pinatta plants may have advantageous traits including: 1) large, bell-shaped flower, 2) ornamental shape of fused sepals, 3) red coloration of the stem, 4) same red coloration on the leaf margin and the middle vein, 5) very strong, upright growth habit and 6) vigorous rooting habit. K. pinatta that should be avoided in a breeding program: include 1) white spotting on the stems, 2) grayish leaf color, 3) poor branching habit, 4) poor production of cuttings and 5) poor post-production longevity.

Advantageous traits obtained from K. laciniata plants include: 1) intense yellow coloration of flower 2) entire, sinuate or parted leaf, 3) shinning texture of leaf 4) good fixation of the leaf to the stem, 5) production of many cuttings and 6) vigorous rooting habit. Traits from K. laciniata that should be avoided in a breeding program include 1) long length of stem, 2) fast and unstable growth habit and 3) poor post-production longevity.

K. diagremontiana plants may have advantageous traits including: 1) long, bell-shaped flowers 2) pink coloration of flower, 3) green color of leaf, 4) lanceolate shape of leaf, 5) red coloration of margin of leaf, 6) shinning texture of leaf and 7) strong, upright growth habit. K. diagremontiana traits that should be avoided in breeding program include: 1) pink coloration on only 1/3 of petal, 2) long length of stem which places inflorescence high above top of plant, 3) marbling of underside of leaf, 4) crispy leaf texture which breaks easily, 5) tall growth habit, and 6) poor post-production longevity.

K. blossfeldiana×K. laciniata interspecific hybrids typically have leaves that are entire, sinuate or parted with erect and large flowers that are white, pink, purple, yellow, orange, and red. K. blossfeldiana×K. laciniata interspecific hybrids are highly branched, relatively compact and under certain conditions the height of the branches may be up to 30-45 cm.

Double-type Kalanchoe interspecific hybrid cultivars are genetically stable, as evidenced by the stability of the double-type trait through both asexual propagation and sexual crosses. The data from numerous crosses with K. blossfeldiana indicate that the double-type trait segregates as a single dominant gene. The double-type trait can be reproducibly and predictably introgressed into diverse Kalanchoe species and genetic backgrounds.

K. blossfeldiana seeds designated ‘MP African’ that produce plants carrying the double-type gene or trait, and which can be used as a male or female parent in crosses with Kalanchoe species to produce double-type Kalanchoe interspecific hybrid cultivars, were deposited at the American Type Culture Collection (ATCC), P.O. Box 1549, Manassas, Va. 20108, U.S.A., and accorded ATCC accession no. ______.

As a first step in making interspecific hybrids of the instant invention, a single or double-type Kalanchoe plant selection is crossed with a single-type Kalanchoe selection from another species. Progeny are screened for fertile selections. Large numbers of progeny may have to be screened to identify fertile selections. The fertile selections may be screened for those exhibiting the double-type flower trait if one of the parents was a double-type selection. Alternatively, the single-type fertile interspecific hybrid is crossed, either as the male or female parent, with a double-type Kalanchoe selection. A double-type hybrid progeny plant with desirable phenotypic characteristics is propagated asexually by conventional methods to determine if the phenotypic characteristics are stable.

For example, a K. blossfeldiana (tetraploid)×K. laciniata (diploid) interspecific hybrid is by nature triploid and thus sterile. K. blossfeldiana×K. laciniata interspecific hybrid progeny plants were screened and ‘Yellow African’, described in U.S. Plant Pat. No. 12,299, was identified. This fertile K. blossfeldiana×K. laciniata interspecific hybrid has been used to breed a series of interspecific hybrid cultivars designated African Treasures™. One such cultivar was designated KJ 2000 0716 and is described in pending U.S. plant patent application Ser. No. 10/654,571, incorporated herein by reference.

KJ 2000 0716 was identified in the progeny originating from a cross between ‘Yellow African’ and a single-type K. blossfeldiana. The three new double-flowered Kalanchoe interspecific hybrids described in Examples 2, 3 and 4 herein originated from crosses between ‘KJ 2000 0716’ as the female parent, and ‘Monroe’ as male double-type K. blossfeldiana parent. ‘Monroe’ is described in U.S. Plant Pat. No. 14,714.

Recurrent selection is used to increase the number of petals per flower found in the Kalanchoe interspecific hybrid plants of the instant invention. A double-type Kalanchoe interspecific hybrid plant is selfed, or crossed to another double-type Kalanchoe plant, and the progeny screened for plants with double-type flowers with an increased number of petals per flower compared to the double-type parents.

1. General Breeding Methods

The Kalanchoe plants of the instant invention were crossed as follows. When the pistil is fully developed and the anthers have not opened, the pistil is to some extent uncovered. During this time the stigma is susceptible to pollination. Pollen is harvested from the male using a pencil or small brush and transferred to the stigma. Once pollination is complete, the seeds develop within the ovary and the pistils are harvested when ripe. The seeds are dried before sowing in peat-based soil mix.

Breeding with double-type K. blossfeldiana requires careful uncovering of the pistil. It is very easy to wound the ovary and thereby hinder germination and development of seeds. In addition, if the female parent is water-stressed during pollination and subsequent seed development, the number of seeds obtained appear to increase.

2. Plant Growth Conditions

The Kalanchoe plants described herein were grown in a greenhouse at 64.4° F. during the day and 68° F. during the night. The plants were produced in pots with a diameter of 10.5 cm or 13 cm. Cuttings were grown under long-day conditions (16 hours light, 8 hours night) during the first 3-8 weeks following planting, depending on cultivar and pot size. Between 4-9 weeks after planting, the plants were transferred to short-day conditions (10 hour light and 14 hour dark). The flowering is induced by short-day conditions. Between 13-19 weeks after planting, depending on cultivar, pot size, and time of year, the plants were mature with flowers that were opening or about to open.

The plants were grown under natural light conditions supplemented with 70 μmol/m2/s SON-T light when the natural light was less than 100 μmol/m2/s. Plants were grown in a peat based soil mix and were watered with a solution containing 200 parts per million (ppm) nitrogen, 200 ppm potassium, 40 ppm phosphorous, 200 ppm calcium, 40 ppm magnesium, 60 ppm sulphate, 1 ppm iron, 0.6 ppm manganese, 0.1 ppm copper, 0.1 ppm zink, 0.3 ppm borium, 0.03 ppm molybdenum.

The following examples are set forth as representative of the specific and preferred embodiments of the present invention. These examples are not to be construed as limiting the scope of the invention in any manner. It should be understood that many variations and modifications can be made while remaining within the spirit and scope of the invention.

### EXAMPLE 1

The data from numerous crosses with K. blossfeldiana indicate that the double-type trait segregates as a single dominant gene. A total of 2,826 progeny from crosses of K. blossfeldiana double-type×K. blossfeldiana single-type have been analyzed. Among these 2,826 progeny, 910 exhibited the double-type trait. Seeds carrying the double-type trait germinate at a lower frequency than seeds carrying the single-type trait. This lower germination frequency has probably impacted the segregation pattern which is not the 1:1 ratio expected for a trait controlled by a single dominant gene. Regardless of what the genetic basis for control of the double-type trait in Kalanchoe might be, these data demonstrate the double-type trait can be reproducibly and predictably introgressed into diverse Kalanchoe genetic backgrounds.

### EXAMPLE 2

### Cultivar ‘KJ 2003 0761’

The new K. blossfeldiana×K. laciniata interspecific hybrid ‘KJ 2003 0761’ was produced using a proprietary single-type selection of K. blossfeldiana×K. laciniata designated ‘KJ 2000 0716’, as the female parent. This female parent is described in pending U.S. plant patent application Ser. No. 10/654,571 and in European Union Community Variety Rights application serial No. 2003/0974, and is the second generation progeny of a fertile interspecific hybrid between K. blossfeldiana and K. laciniata. The male parent was a K. blossfeldiana double-type selection designated ‘Monroe’, and is described in U.S. Plant Pat. No. PP14,714. The new Kalanchoe cultivar ‘KJ 2003 0761’ was discovered and selected as a flowering plant within the progeny of the stated cross in a controlled environment in Hinnerup, Denmark.

Asexual reproduction of the new cultivar by vegetative terminal cuttings was first performed in February, 2004, in Hinnerup, Denmark, and has demonstrated that the combination of characteristics as herein disclosed for the new cultivar are firmly fixed and retained through successive generations of asexual reproduction. The new cultivar reproduces true-to-type through asexual reproduction.

The following traits have been repeatedly observed and are determined to be basic characteristics of the new Kalanchoe cultivar ‘KJ 2003 0761’ which in combination distinguish this Kalanchoe as a new and distinct cultivar:
1. large number of petals per flower resulting in a double-type trait;
2. the large size of petals resulting in a large flower;
3. the hastate, dissected leaves on young as well as mature leaves;
4. the intense sunny-yellow color of petals; and
5. the large number of flowers per plant.

The new Kalanchoe cultivar ‘KJ 2003 0761’ has not been observed under all possible environmental conditions. The phenotype of the new cultivar may vary significantly with variations in environment such as temperature, light intensity, and day length without any change in the genotype of the plant. The following observations, measurements and values describe the new Kalanchoe cultivar ‘KJ 2003 0761’ grown in Hinnerup, Denmark, under conditions which closely approximate those generally used in commercial practice.

Plants of ‘KJ 2003 0761’ differ from plants of the parental cultivars in the following characteristics:

| | New Cultivar | Female Parent | Male Parent |
|---|---|---|---|
| Trait | ‘KJ 2003 0761’ | ‘KJ 2000 0716’ | ‘Monroe’ |
| Height of cultivar | About 25 cm | About 38 cm | About 24 cm |
| Flower Type | Double-type | Single-type | Double-type |
| Flower Diameter | 18-22 mm | 20-25 mm | 16-19 mm |
| Flower number | 250-300 | 500 | 350-400 |
| Number of | Up to 16 full or partial | 4 | Up to 27 full or |
| Corolla Lobes | petals | | partial petals. |
| Corolla | From outer to center | Red-orange, | Up to 27 full or |
| Coloration | of flower: | RHS 44B | partial petals |
| | First circle | | |
| | Yellow, RHS 13D | | |
| | Next circle | | |
| | Yellow-orange, RHS | | |
| | 15D | | |
| | Center circle | | |
| | Yellow, RHS 13A | | |
| | Whole flower faints to | | |
| | light yellow-orange, | | |
| | RHS 21B | | |
| Shape of petal | The width of the petal | The width of the | The width of the |
| | is 7 mm. The length is | petal is 10 mm. | petal is 4 mm. The |
| | 8 mm. The shape | The length is 13 mm. | length is 8 mm. |
| | ovate with mucronate | The shape is | The shape is ovate |
| | apex. | obovate with | with cuspidate tip. |
| | | cuspidate tip. | |
| Leaf | Hastate, dissected | Hastate, dissected | Obovate leaves |
| | leaves with a long | leaves with a long | with obtuse leaf |
| | petiole and long | petiole and long | tips and truncate |
| | lamina. The leaf apex | lamina. The leaf | bases. The leaf |
| | is obtuse and the base | apex is obtuse and | margin is |
| | is cunate. | the base is cunate. | undulate. |
| Leaf texture | The foliage is | The foliage is | The foliage is |
| | glabrous and shinning | glabrous, and more | glabrous and |
| | both above and below. | shinning above | shinning both |
| | | than below, | above and below. |

Of the many commercial cultivars known to the present Inventors, the most similar in comparison to ‘KJ 2003 0761’ is parental cultivar ‘KJ 2000 0716’. Comparing these two cultivars, the main distinction is the form and the color of the flower, as described in the preceding table.

In the following description, color references are made to the Royal Horticultural Society Colour Chart (RHS), published 1986, except where general colors of ordinary significance are used. Color values were taken under daylight conditions at approximately 12 a.m. in a greenhouse in Hinnerup, Denmark. The age of the plant described is 15 weeks (from the time the cutting was planted in growth medium to when the picture was taken).

PARENTAGE:

Male or pollen parent: K. blossfeldiana cultivar ‘Monroe’ (disclosed in U.S. Plant Pat. No. PP 14,714)

Female or seed parent: K. blossfeldiana×K. laciniata interspecific hybrid cultivar ‘KJ 2000 0716’ (disclosed in U.S. patent application Ser. No. 10/654,571)

CLASSIFICATION:

Botanical: K. blossfeldiana×K. laciniata interspecific hybrid cultivar ‘KJ 2003 0761’ (2nd generation after K. blossfeldiana×K. laciniata interspecific hybrid back-crossed with K. blossfeldiana).

PROPAGATION: Vegetative Terminal Cuttings.

Rooting Habit: The cultivar has numerous, branched, fine and fibrous roots.

Time to initiate roots: It takes between one and two weeks to initiate roots.

Time to produce a rooted cutting: It will take three weeks to produce a well rooted cutting. In winter time, it can take on week more.

PLANT: If the plants are grown according to the description above, it will perform as described below. Variation from this should be expected over the course of the year, and if the physical growing conditions varies from the description.

Growth habit: This cultivar is upright and uniform. The flowers are formed above the top of the leaf canopy, but the flowers will also appear between the leaves. The flowers are assembled in numerous compound inflorescences ‘KJ 2003 0761’ is freely flowering with numerous compound cymes.

Growth rate: Ready for sale after 16 weeks.

Branching habit and description: Freely branching; typically 8 to 12 lateral branches develop per plant. Pinching (removal of the terminal apex) is not mandatory, but will enhance lateral branching. When grown in 10 cm pots the cultivar is not pinched.

Height at flowering: The height of the cultivar is about 25 cm from the bottom of the pot to the top of the plant, depending on the growth conditions.

Spread/Diameter at flowering: The diameter of the cultivar at flowering is about 25 cm.

STEMS:

Appearance: The cultivar has around 10 stems with none, one or numerous leaf pairs on the stem. The inflorescence on the top of the flower stem is branched giving an inverted triangle with a rounded crown. Each branch has between 30 and 70 flowers.

Aspect: The stem is strong.

Length: The length varies from 10 to 15 cm depending on growth conditions.

Texture: The texture of the stem is glabrous and shinning.

Color: The color of the stem is dark green, RHS 137 B.

FOLIAGE:

Arrangement: The foliage is yellow-green with the same color of the immature as well as the mature leaf, RHS 147 A. The majority of the leaves are at the base of the plant, but immature leaves appear also on the flowering stem.

Overall Shape of Leaf: The leaves are defined as hastate, dissected leaves.
Apex: The form of the apex is obtuse.
Base: The form of the base is cunate.

Length: The length of the foliage varies from 6 cm for an immature leaf to 12 cm for a mature leaf.

Width: The width varies from 3 cm for an immature leaf to 9 cm for a mature leaf.

Margin: The foliage has a dentate margin.

Texture: The foliage is glabrous and shinning.

Color of Upper Surface:
Mature leaf: The color of the mature leaf is a dark yellow-green color, and is closely described with RHS 147 A, though it is a little greener.
Immature leaf: The immature leaf has the same color as the mature leaf, a dark yellow-green color, and is closely described with RHS 147 A, though it is a little greener.

Color of Lower Surface:
Mature leaf: The color of the lower surface is dark yellow-green and is closely describe with RHS 147 A.
Immature leaf: The immature leaf has the same color as the mature leaf and is defined with RHS 147 A.

Venation Color: There is no visual appearance of veins.
Upper surface: No difference between the color of venation and the rest of the leaf.
Lower surface: No difference between the color of venation and the rest of the leaf.

Petiole:
Length: The length of the petiole varies between an immature and mature leaf. The length of an immature leaf is about 0.75 cm. The length of a mature leaf is about 3 cm.
Diameter: The diameter of the petiole varies between an immature and mature leaf. The diameter of an immature leaf is 5 mm. The diameter of a mature leaf is 9 mm.
Color: Yellow-green, between RHS 147 A and 147 B.

FLOWER DESCRIPTION:

Flower type and habit: The flower is a double-type with up to 16 petals per flower. The average number of petals per flower is 12.

Natural flowering season: The flowering season is year round, when grown according to description above. When planting outside the flowering season will be in the summer (April to October), depending on local climatic conditions.

Time to flower: The time from start of short day treatment to 1st flower is about 66 days, approximately 1 week longer in winter time.

Flowering stem length: The length of the flowering stem is ranging from 13 to 14 cm depending on the growth conditions.

Post-production longevity: ‘KJ 2003 0761’ maintains good leaf and flower display for at least 6 weeks under interior environmental conditions.

Winter Hardiness/weather tolerance: ‘KJ 2003 0761’ withstands wind, rain and direct sunlight, and can cope with temperatures between 40 and 95 degrees F.

Fragrance: ‘KJ 2003 0761’ has no fragrance.

Flower size: The diameter of the flower range from 18 to 22 mm.

Overall shape: The shape is rose-like, defined by at least 5 petals per flower.

Quantity: The cultivar produces a large number of flower ranging from 250 to 300 flowers per plant.

Bud:
Rate of opening: From the bud is showing color the flower will open within 10 days.
Color: The color of the bud is yellow-green, RHS 145 B.
Shape: The shape of the bud is ellipsoidal with the smallest diameter toward the base.
Length: The length of the bud is about 1.3 cm.
Diameter: The diameter of the bud is about 3 mm at the base and about 5 mm at the top.

Petal:
Quantity: Typically 13-16 petals fused at the base.
Shape: The shape of petal is oval.
Length: The length is about 7 mm.
Width: The width is about 8 mm.
Apex: The shape of the apex is mucronate.
Margin: The shape of the margin can be described as entire.
Texture: The texture of the petals is soft, smooth and mat.
Color when opening:
   Upper surface: The upper surface of the petal is yellow-orange, RHS 17 C.
   Lower surface: The lower surface of the petal is yellow-orange, RHS 16A, with a twist of orange-red, RHS 30 D.
Color when fully open:
   Upper surface: The upper surface of the petal is yellow-orange, RHS 15 A.
   Lower surface: The lower surface the petal is yellow-orange, RHS 16 A, with a twist of orange-red, RHS 30 D.

Sepal:
Quantity: Typically 4 sepals fused at the base.
Shape: The shape of sepal is lineal lanceolate.
Length: The length is 11 mm.
Width: The width is 2 mm.
Apex: The shape of the apex is acute.
Margin: The shape of the margin is entire.
Texture: The texture of the sepals is glabrous and shinning.
Color when Opening:
   Upper surface: The upper surface of the sepal is green, close to RHS 146 A, but a little greener.
   Lower surface: The lower surface of the sepal is green, close to RHS 146A, but a little greener.
Color when Fully Open:
   Upper surface: The upper surface of the sepal is green, close to RHS 146 A, but a little greener.
   Lower surface: The lower surface the sepal is green, close to RHS 146 A, but a little greener.

Peduncle Description:
Length: Every flower has a peduncle with a length of 3 mm.
Color: The peduncle is dark green, RHS 137 B.
Texture: The peduncle is glabrous and shinning.

REPRODUCTIVE ORGANS:

Stamen:
Number: The flower has between 8 and 12 ten stamens.
Color: The color of the stamens is greyed-orange, RHS 177B.

Anthers:
Number: The number of anthers is between 8 and 12.
Size: The size of 1 mm in length.
Color: The color of the anthers is yellow-brown, RHS 14B.

Filament Color: The color of the filament is yellow-green, RHS 145C.

Pollen Color: The color of the pollen is yellow, RHS 17C.

Pollen Amount: A high amount of pollen with a high pollen fertility of 95%, depending on the time of year.

Pistil:
Number: The number of pistils is four.

Stigma:
Shape: The shape of stigma is round.
Color: The color of the stigma is yellow-green, RHS 145A.

Style:
Shape: The shape of the style is thin and cylindrical.
Color: The color of style is yellow-green, RHS 145 A.

Ovary:
Color: The color of ovary is yellow-green, best described between RHS 147A and 147B.

Seeds:
Number: Between 50 and 60 potential seeds per ovary.
Width: The width of the seeds is less than 0.5 mm.
Length: The length of the seeds is less than 1 mm.
Shape: The shape of the seeds is ellipsoidal.
Color: The color of the potential seeds is green, RHS 145 C.

Fruit (Ovary):
Shape: The shape of the fruit is cylindrical.
Width: The width of the fruit is 1 mm.
Length: The length of the fruit is 6 mm.
Color: The color of the fruit is yellow-green, RHS 145 A.

DISEASE RESISTANCE/SUSCEPTIBILITY: No information on disease resistance or susceptibility is currently available.

### EXAMPLE 3

### Cultivar ‘KJ 2003 0638’

The new K. blossfeldiana×K. laciniata interspecific hybrid ‘KJ 2003 0638’ was produced using a proprietary single-type selection of K. blossfeldiana×K. laciniata designated ‘KJ 2000 0716’, as the female parent. This female parent is described in pending U.S. plant patent application Ser. No. 10/654,571 and in European Union Community Variety Rights application serial No. 2003/0974, and is the second generation progeny of a fertile interspecific hybrid between K. blossfeldiana and K. laciniata. The male parent was a K. blossfeldiana double-type selection designated ‘Monroe’ and is described in U.S. Plant Pat. No. PP14,714. The new Kalanchoe cultivar ‘KJ 2003 0638’ was discovered and selected as a flowering plant within the progeny of the stated cross in a controlled environment in Hinnerup, Denmark.

Asexual reproduction of the new cultivar by vegetative terminal cuttings was first performed in February, 2004, in Hinnerup, Denmark, and has demonstrated that the combination of characteristics as herein disclosed for the new cultivar are firmly fixed and retained through successive generations of asexual reproduction. The new cultivar reproduces true-to-type through asexual reproduction.

The following traits have been repeatedly observed and are determined to be basic characteristics of the new Kalanchoe cultivar ‘KJ 2003 0638’ which in combination distinguish this Kalanchoe as a new and distinct cultivar:
1. large number of petals per flower resulting in a double-type or multi-petalled trait;
2. large size of petals resulting in a large flower;
3. the hastate, dissected leaves on young as well as mature leaves;
4. the different soft-white and creamy colors of petals and
5. large number of flowers per plant.

The new Kalanchoe cultivar ‘KJ 2003 0638’ has not been observed under all possible environmental conditions. The phenotype of the new cultivar may vary significantly with variations in environment such as temperature, light intensity, and day length without any change in the genotype of the plant. The following observations, measurements and values describe the new Kalanchoe cultivar ‘KJ 2003 0638’ as grown in Hinnerup, Denmark, under conditions which closely approximate those generally used in commercial practice.

Plants of ‘KJ 2003 0638’ differ from plants of the parental cultivars in the following characteristics:

| | New Cultivar | Female Parent | Male Parent |
|---|---|---|---|
| Trait | ‘KJ 2003 0638’ | ‘KJ 2000 0716’ | ‘Monroe’ |
| Height of cultivar | About 31 cm | About 38 cm | About 24 cm |
| Flower Type | Double-type | Single-type | Double-type |
| Flower Diameter | 20-25 mm | 20-25 mm | 16-19 mm |
| Flower Number | 250-300 | 500 | 350-400 |
| Per Plant | | | |
| Number of | Up to 17 full or partial | 4 | Up to 27 full or |
| Corolla Lobes | petals | | partial petals |
| Corolla | From outer to center | Red-orange, | White, RHS 155 D |
| Coloration | of flower: | RHS 44B | |
| | First circle | | |
| | Yellow-orange, RHS | | |
| | 20D | | |
| | Next circle | | |
| | Yellow-orange, RHS | | |
| | 19B | | |
| | Center circle | | |
| | Yellow-orange, RHS | | |
| | 20D with a twist of | | |
| | red, RHS 39C | | |
| | Whole flower faints to | | |
| | light orange RHS 27C | | |
| Shape of petal | The width of the petal | The width of the | The width of the |
| | is 9 mm. The length is | petal is 10 mm. | petal is 4 mm. The |
| | 6 mm. The shape is | The length is 13 mm. | length is 8 mm. |
| | ovate with mucronate | The shape is | The shape is ovate |
| | apex. | obovate with | with cuspidate tip. |
| | | cuspidate tip. | |
| Leaf | Hastate, dissected | Hastate, dissected | Obovate leaves |
| | leaves with a long | leaves with a long | with obtuse leaf |
| | petiole and long | petiole and long | tips and truncate |
| | lamina. The leaf apex | lamina. The leaf | bases. The leaf |
| | is obtuse and the base | apex is obtuse and | margin is |
| | is cunate. | the base is cunate. | undulate. |
| Leaf texture | The foliage is | The foliage is | The foliage is |
| | glabrous and shinning | glabrous and more | glabrous and |
| | both above and below. | shinning above | shinning both |
| | | than below. | above and below. |

Of the many commercial cultivars known to the present Inventors, the most similar in comparison to ‘KJ 2003 0638’ is parental cultivar ‘KJ 2000 0716’. Comparing these two cultivars, the main distinction is the form and the color of the flower, as described in the preceding table.

‘KJ 2003 0638’ has not been observed under all possible environmental conditions. The phenotype of the new cultivar may vary significantly with variations in environment such as temperature, light intensity, and day length without any change in the genotype of the plant.

The aforementioned photographs, together with the following observations, measurements and values describe the new Kalanchoe cultivar as grown in a greenhouse in Hinnerup, Denmark, under conditions which closely approximate those generally used in commercial practice.

In following description, color references are made to the Royal Horticultural Society Colour Chart (RHS), published 1986, except where general colors of ordinary significance are used. Color values were taken under daylight conditions at approximately 12 a.m. in a greenhouse in Hinnerup, Denmark. The age of the plant described is 15 weeks (from the time the cutting was planted in growth medium to when the picture was taken).

PARENTAGE:

Male or pollen parent: K. blossfeldiana cultivar ‘Monroe’ (disclosed in U.S. Plant Pat. No. PP14,714)

Female or seed parent: K. blossfeldiana×K. laciniata interspecific hybrid cultivar ‘KJ 2000 0716’ (disclosed in U.S. patent application Ser. No. 10/654,571)

CLASSIFICATION:

Botanical: K. blossfeldiana×K. laciniata interspecific hybrid cultivar ‘KJ 2003 0638’ (2nd generation after K. blossfeldiana×K. laciniata interspecific hybrid back-crossed with K. blossfeldiana).

PROPAGATION: Vegetative terminal cuttings

Rooting Habit: The cultivar has numerous, branched, fine and fibrous roots.

Time to initiate roots: It takes between one and two weeks to initiate roots.

Time to produce a rooted cutting: It will take three weeks to produce a well rooted cutting. In winter time, it can take one week more.

PLANT: If the plants are grown according to the description above, it will perform as described below. Variation from this should be expected over the course of the year, and if the physical growing conditions varies from the description.

Growth Habit: This cultivar is upright and uniform. The flowers are formed above the top of the leaf canopy, but flowers will also appear between the leaves. The flowers are assembled in numerous compound inflorescences.

Growth rate: Ready for sale after 16 weeks.

Branching habit and description: Freely branching; typically 4 to 8 lateral branches develop per plant. Pinching (removal of the terminal apex) is not mandatory, but will enhance lateral branching. When grown in 10 cm pots the cultivar is not pinched.

Height at flowering: The height of the cultivar is about 31 cm, from the bottom of the pot to the top of the plant, depending on growth conditions.

Spread/Diameter at flowering: The diameter at flowering is about 30 cm.

STEMS:

Appearance: The cultivar has around 8 stems with none, one or numerous leaf pairs on the stem. The inflorescence on the top of the flower stem is branched, giving an inverted triangle with a rounded crown. Each branch has between 40 and 60 flowers.

Aspect: The stem is strong.

Length: The length of the stem is about 14 cm depending on growth conditions.

Texture: The texture of the stem is glabrous and shinning.

Color: The color of the stem is yellow-green, RHS 146 A.

FOLIAGE:

Arrangement: The foliage is yellow-green with the same colors of the immature as well as the mature leaf, RHS 146 A and RHS 147 A. The majority of the leaves are at the base of the plant, but immature leaves appear also on the flowering stem.

Overall Shape of Leaf: The leaves are defined as hastate, dissected leaves.

Apex: The form of the apex is obtuse.

Base: The form of the base is cunate.

Length: The length of the foliage varies from about 3 cm for an immature leaf to about 14 cm for a mature leaf.

Width: The width varies from about 2 cm for an immature leaf to about 13 cm for a mature leaf.

Margin: The foliage has a dentate margin.

Texture: The foliage is glabrous and shinning both above and below.
Color of Upper Surface:
   Mature leaf: The color of the mature leaf is dark yellow-green and is closely described with RHS 147 A.
   Immature leaf: The immature leaf has the same color as the mature leaf, dark yellow-green, described with RHS 147 A.
Color of Lower Surface:
   Mature leaf: The color of the lower surface is different from the upper surface, and is closely described with a dark yellow-green color between RHS 146 A and RHS 147 A.
   Immature leaf: The immature leaf has the same color as the mature leaf, dark yellow-green, and is closely described with a dark yellow-green color between RHS 146 A and RHS 147 A.

Venation Color: There is no visual appearance of veins.
Upper surface: There is no difference between the color of venation and the rest of the leaf.
Lower surface: There is no difference between the color of venation and the rest of the leaf.

Petiole:
Length: The length of the petiole varies between an immature and mature leaf. The length of an immature leaf varies between 0.5 and 1 cm. The length of a mature leaf varies between 3 and 3.5 cm.
Diameter: The diameter of petiole varies between an immature and mature leaf. The diameter of an immature leaf is 4 mm. The diameter of a mature leaf is 5 mm.
Color: There is no difference between the color of petiole and the rest of the leaf.

FLOWER DESCRIPTION:

Flower type and habit: The flower type is a double-type with 16 to 19 petals per flower. The average number of petals per flower is 17.

Natural flowering season: The flowering season is year round, when grown according to description above. When planting outside the flowering season will be in the summer (April to October), depending on local climatic conditions.

Time to flower: The time from start of short day treatment to 1^{st} flower is about 74 days, approximately 1 week longer in winter time.

Flowering stem length: The length of the flowering stem is ranging from 13 to 14 cm depending on the growth conditions.

Post-production longevity: ‘KJ 2003 0638’ maintains good leaf and flower display for at least 6 weeks under interior environmental conditions.

Winter Hardiness/weather tolerance: ‘KJ 2003 0638’ withstands wind, rain and direct sunlight, and can cope with temperatures between 40 and 95 degrees F.

Fragrance: ‘KJ 2003 0638’ has no fragrance.

Flower size: The diameter of the flower range from is 2.5 to 3 cm.

Overall shape: The shape is rose-like, defined by at least 5 petals per flower.

Quantity: The cultivar produces a large number of flower ranging from 200 to 250 flowers per plant.

Bud:
Rate of opening: From the bud is showing color the flower will open within 10 days.
Color: At the tip the bud is yellow-green, RHS 145 B, with a twist of orange 24 A. At the base the bud has the same yellow-green color, RHS 145B.
Shape: The shape of the bud is ellipsoidal with the smallest diameter towards the base.
Length: The length of the bud is about 1.5 cm.
Diameter: The diameter of the bud is about 8 mm at the tip and about 4 mm at the base.

Petal:
Quantity: Typically 16-17 fused at the base.
Shape: The shape of petal is oval.
Length: The length is about 9 mm.
Width: The width is about 6 mm.
Apex: The shape of apex is mucronate.
Margin: The shape of the margin can be described as entire.
Texture: The texture of the petals is soft, smooth and mat.
Color when opening:
   Upper surface: The color of the upper surface of the petals can be described as follows: Going from the outer circle to the inner circle of petals, the first circle of petals is yellow-orange, RHS 20 D, the next is yellow-orange, RHS 19 B, and the middle is yellow-orange, RHS 20 B, with a touch of red, RHS 39 C.
   Lower surface: The color of the lower surface of the petals is yellow-orange, RHS 23 D, with a touch of red, RHS 38 A.
Color when fully open:
   Upper surface: At maturity the upper surface of the whole flower fades to orange RHS 27 C.

Lower surface: At maturity the lower surface of the whole flower fades to orange RHS 27 C.

Sepal:
Quantity: Typically 4 sepals fused at the base.
Shape: The shape of sepal is lanceolate.
Length: The length is 10 mm.
Width: The width is 3 mm.
Apex: The shape of the apex is acute.

Margin: The shape of the margin is entire.

Texture: The texture of the sepals is glabrous and shinning.
Color when opening:
   Upper surface: The upper surface of the sepal is green, RHS 137 B.
   Lower surface: The lower surface of the sepal is green,

RHS 137 B.
Color when fully open:
   Upper surface: The upper surface of the sepal is green, RHS 137 B.
Lower surface: The lower surface the sepal is green, RHS 137B.

Peduncle:
Length: Every flower has a peduncle with a length of 5 mm.
Color: The peduncle is yellow-green; RHS 146 A.
Texture: The peduncle is glabrous and shinning.

REPRODUCTIVE ORGANS:

Stamen:
Number: The flower has between 7 and 9 stamens.
Color: The color of the stamens is greyed-orange, RHS 177 B.

Anthers:
Number: The number of anthers is 7 and 9.
Size: The anthers are less than 1 mm in length.
Color: The color of the anthers is yellow-brownish, RHS 14 B.

Filament Color: The color of the filament is yellow-green, RHS 145 C.

Pollen Color: The color of the pollen is yellow, RHS 17 C.

Pollen Amount: A medium amount of pollen with very good pollen fertility of 100% depending on time of year.

Pistil:
Number: The number of pistils is four.

Stigma:
Shape: The shape of the stigma is round.
Color: The color of the stigma is yellow-green, RHS 145 A.

Style:
Shape: The shape of the style is thin and cylindrical.
Color: The color of the style is yellow-green, RHS 145 A.

Ovary:
Color: The color of ovary is green, RHS 144 B.

Seeds:
Number: Between 50 and 60 potential seeds per ovary.
Width: The width of the seeds is less than 0.5 mm.
Length: The length of the seeds is less than 1 mm.
Shape: The shape of the seeds is ellipsoidal.
Color: The color of the potential seeds is green, RHS 145 C.

Fruit (Ovary):
Shape: The shape of the fruit is cylindrical.
Width: The width of the fruit is 1 mm.
Length: The length of the fruit is 6 mm.
Color: The color of the fruit is yellow green, RHS 145 A.

DISEASE RESISTANCE/SUSCEPTIBILITY: No information on disease resistance or susceptibility is currently available.

### EXAMPLE 4

### Cultivar ‘KJ 2003 0747’

The new K. blossfeldiana×K. laciniata interspecific hybrid ‘KJ 2003 0747’ was produced using a proprietary single-type selection of K. blossfeldiana×K. laciniata designated ‘KJ 2000 0716’, as the female parent. This female parent is described in pending U.S. plant patent application Ser. No. 10/654,571 and in European Union Community Variety Rights application serial No. 2003/0974, and is the second generation progeny of a fertile interspecific hybrid between K. blossfeldiana and K. laciniata. The male parent was a K. blossfeldiana double-type selection designated ‘Monroe’ and is described in U.S. Plant Pat. No. PP14,714. The new Kalanchoe cultivar ‘KJ 2003 0747’ was discovered and selected as a flowering plant within the progeny of the stated cross in a controlled environment in Hinnerup, Denmark.

Asexual reproduction of the new cultivar by vegetative terminal cuttings was first performed in February, 2004, in Hinnerup, Denmark, and has demonstrated that the combination of characteristics as herein disclosed for the new cultivar are firmly fixed and retained through successive generations of asexual reproduction. The new cultivar reproduces true-to-type through asexual reproduction.

The following traits have been repeatedly observed and are determined to be basic characteristics of the new Kalanchoe cultivar ‘KJ 2003 0747’ which in combination distinguish this Kalanchoe as a new and distinct cultivar:
1. large number of petals per flower resulting in a double-type or multi-petalled trait;
2. large size of petals resulting in a large flower;
3. hastate, dissected leaves on young as well as mature leaves;
4. the different yellow and soft-white colors of petals and
5. large number of flowers per plant.

The new Kalanchoe cultivar ‘KJ 2003 0747’ has not been observed under all possible environmental conditions. The phenotype of the new cultivar may vary significantly with variations in environment such as temperature, light intensity, and day length without any change in the genotype of the plant. The following observations, measurements and values describe the new Kalanchoe cultivar ‘KJ 2003 0747’ as grown in Hinnerup, Denmark under conditions which closely approximate those generally used in commercial practice.

Plants of ‘KJ 2003 0747’ differ from plants of the parental cultivars in the following characteristics:

| | New Cultivar | Female Parent | Male Parent |
|---|---|---|---|
| Trait | ‘KJ 2003 0747’ | ‘KJ 2000 0716’ | ‘Monroe’ |
| Height of | About 31 cm | About 38 cm | About 24 cm |
| Cultivar | | | |
| Flower Type | Double-type | Single-type | Double-type |
| Flower Diameter | 20-25 mm | 20-25 mm | 16-19 mm |
| Flower Number | 250-300 | 500 | 350-400 |
| Per Plant | | | |
| Number of | Up to 25 full or partial | 4 | Up to 27 full or |
| Corolla Lobes | petals | | partial petals |
| Corolla Coloration | From outer petals to | Red-orange, | White, RHS 155 D |
| | center of flower: | RHS 44B | |
| | First circle | | |
| | Yellow, RHS 13D | | |
| | Next circle | | |
| | Yellow-orange, RHS | | |
| | 15D | | |
| | Center circle | | |
| | Yellow, RHS 13A | | |
| | with a touch of RHS | | |
| | 24C | | |
| | Whole flower fades to | | |
| | light yellow (RHS | | |
| | 15D) as matures | | |
| Shape of Petal | The width of the petal | The width of the | The width of the |
| | is 8 mm. The length is | petal is 10 mm. The | petal is 4 mm. The |
| | 10 mm. The shape | length is 13 mm. | length is 8 mm. |
| | ovate with mucronate | The shape is | The shape is ovate |
| | apex. | obovate with | with cuspidate tip. |
| | | cuspidate tip. | |
| Leaf | Hastate, dissected | Hastate, dissected | Obovate leaves |
| | leaves with a long | leaves with a long | with obtuse leaf |
| | petiole and long | petiole and long | tips and truncate |
| | lamina. The leaf apex | lamina. The leaf | bases. The leaf |
| | is obtuse and the base | apex is obtuse and | margin is undulate |
| | is cunate. | the base is cunate. | |
| Leaf Texture | The foliage is | The foliage is | The foliage is |
| | glabrous and shinning | glabrous, and more | glabrous and |
| | both above and below. | shinning above | shinning both |
| | | than below. | above and below. |

Of the many commercial cultivars known to the present Inventors, the most similar in comparison to ‘KJ 2003 0747’ is parental cultivar ‘KJ 2000 0716’.

Comparing these two cultivars, the main distinctions are the form and the color of the flower, as described in the preceding table.

The new Kalanchoe cultivar ‘KJ 2003 0747’ has not been observed under all possible environmental conditions. The phenotype of the new cultivar may vary significantly with variations in environment such as temperature, light intensity, and day length without any change in the genotype of the plant.

The aforementioned photographs, together with the following observations, measurements and values describe the new Kalanchoe cultivar as grown in a greenhouse in Hinnerup, Denmark, under conditions which closely approximate those generally used in commercial practice.

In the following description, color references are made to the Royal Horticultural Society Colour Chart (RHS), published 1986, except where general colors of ordinary significance are used. Color values were taken under daylight conditions at approximately 12 a.m. in a greenhouse in Hinnerup, Denmark. The age of the plant described is 15 weeks (from the time the cutting was planted in growth medium to when the picture was taken).

PARENTAGE:

Male or pollen parent: K. blossfeldiana cultivar ‘Monroe’ (disclosed in U.S. Plant Pat. No. PP14,714)

Female or seed parent: K. blossfeldiana×K. laciniata interspecific hybrid cultivar ‘KJ 2000 0716’ (disclosed in U.S. patent application Ser. No. 10/654,571)

CLASSIFICATION:

Botanical: K. blossfeldiana×K. laciniata interspecific hybrid cultivar ‘KJ 2003 0747’ (2nd generation after K. blossfeldiana×K. laciniata interspecific hybrid back-crossed with K. blossfeldiana).

PROPAGATION: Vegetative terminal cuttings

Rooting Habit: The cultivar has numerous, branched, fine and fibrous roots.

Time to initiate roots: It takes between one and two weeks to initiate roots.

Time to produce a rooted cutting: It will take three weeks to produce a well rooted cutting. In winter time, it can take one week more.

PLANT: If the plants are grown according to the description above, it will perform as described below. Variation from this should be expected over the course of the year, and if the physical growing conditions varies from the description.

Growth Habit: This cultivar is upright and uniform. The flowers are formed above the leaf canopy, but flowers will also appear between the leaves. The flowers are assembled in numerous compound inflorescences.

Growth rate: Ready for sale after 14 weeks.

Branching habit and description: Freely branching; typically 4 to 8 lateral branches develop per plant. Pinching (removal of the terminal apex) is not mandatory, but will enhance lateral branching. When grown in 10 cm pots the cultivar is not pinched. ‘KJ 2003 0747’ grown in 13 cm pot is pinched over 4 leaf pair. The enhancement of lateral branching in relation to the season varies.

Height at flowering: The height of the cultivar is about 40 cm, from the bottom of the pot to the top of the plant, depending on growth conditions.

Spread/Diameter at flowering: The diameter at flowering is about 25 cm.

STEMS:

Appearance: The cultivar has around 8 stems with none, one or numerous leaf pairs on the stem. The inflorescence on top of the flower stem is branched, giving an inverted triangle with a rounded crown. Each inflorescence has between 40 and 60 flowers.

Aspect: The stem is strong.

Length: The length of the stem is between 15 and 20 cm depending on growth conditions

Texture: The texture of the stem is glabrous and shinning.

Color: The color of the stem is yellow-green, RHS 146 A.

FOLIAGE:

Arrangement: The foliage is yellow-green with the same colors of the immature as well as the mature leaf, RHS 146 A and RHS 147 A. The majority of the leaves are at the base of the plant, but immature leaves appear also on the flowering stem.

Overall Shape of Leaf: The leaves are defined as hastate, dissected leaves.

Apex: The form of the apex is obtuse.

Base: The form of the base is cunate.

Length: The length of the foliage varies from about 6 cm for an immature leaf to about 22 cm for a mature leaf.

Width: The width varies from about 3 cm for an immature leaf to about 13 cm for a mature leaf.

Margin: The foliage has a dentate margin.

Texture: The foliage is glabrous and shinning both above and below.

Color of Upper Surface:
Mature leaf: The color of the mature leaf is dark yellow-green, and is closely described with RHS 147 A.
Immature leaf: The immature leaf has the same color as the mature leaf, dark yellow-green color, described with RHS 147 A.

Color of Lower Surface:
Mature leaf: The color of the lower surface is different from the upper surface, dark yellow-green color, and is closely described with RHS 146 A.
Immature leaf: The immature leaf has the same color as the mature leaf, and is defined with dark yellow-green color, RHS 146 A.

Venation Color: There is no visual appearance of veins.
Upper surface: There is no difference between the color of venation and the rest of the leaf.
Lower surface: There is no difference between the color of venation and the rest of the leaf.

Petiole:

Length: The length of petiole varies between an immature and mature leaf. The length of an immature leaf varies between 1.5 and 2 cm. The length of a mature leaf varies between 5 and 8 cm.

Diameter: The diameter of petiole varies between an immature and mature leaf. The diameter of an immature leaf is 5 mm. The diameter of a mature leaf is 8 mm.

Color: There is no difference between the color of petiole and the rest of the leaf.

FLOWER DESCRIPTION:

Flower type and habit: The flower type is a double-type with 16 to 25 petals per flower. The average number of petals per flower is 19.

Natural flowering season: The flowering season is year round, when grown according to description above. When planting outside the flowering season will be in the summer (April to October), depending on local climatic conditions.

Time to flower: The time from start of short day treatment to 1st flower is about 67 days, approximately 1 week longer in the winter time.

Flowering stem length: The length of the flowering stem is between 14 and 20 cm depending of the growth conditions.

Post-production longevity: ‘KJ 2003 0747’ maintains good leaf and flower display for at least 5 weeks under interior environmental conditions

Winter Hardiness/weather tolerance: ‘KJ 2003 0747’ withstands wind, rain and direct sunlight, and can cope with temperatures between 40 and 95 degree F.

Fragrance: ‘KJ 2003 0747’ has no fragrance.

Flower size: The diameter of the flower ranges from 1.8 to 3.0 cm.

Overall shape: The shape is rose-like defined by at least 5 petals per flower.

Quantity: The cultivar produces a large number of flowers ranging from 250 to 300 flowers per plant.

Bud:
Rate of opening: From the bud is showing color the flower will open within 10 days.
Color: At the tip the bud is yellow, RHS 12 A, with a twist of yellow-orange, RHS 23 B, at the edge. At the base the bud is yellow-green and can closely be describe with RHS 144 B.
Shape: The shape of the bud is ellipsoidal with the smallest diameter toward the base.
Length: The length of the bud is about 1.7 cm
Diameter: The diameter of the bud is about 8 mm at the top and about 4 mm at the base.

Petal:
Quantity: Typically 16-20 petals fused at the base.
Shape: The shape of petal is oval.
Length: The length is 1 cm.
Width: The width is 7 mm.
Apex: The shape of apex is mucronate.
Margin: The shape of the margin can be described as entire.
Texture: The texture of the petals is soft, smooth and mat.
Color when opening:
   Upper surface: The upper surface of the petal has different yellow colors changing from soft yellow to warm yellow, going from the outer circle to the center circle of the petals. The first petals are yellow, RHS 13 D, the next are yellow-orange, RHS 15 D, and the middle are yellow, RHS 13 A with a touch of 24C.
   Lower surface: As for the lower surface the soft light yellow colors of the petals also changes going from the outer circle to the center circle of the petals. The first petals are yellow, RHS 6 D with a touch of RHS 29 D, the next are yellow, RHS 10 D and the center is yellow, RHS 4 D.
Color when fully open:
   Upper surface: At maturity the whole flower fades to orange, RHS 27 D, with a touch of RHS 29 D in the center of the flower.
   Lower surface: At maturity the lower surface fades to orange RHS 27 D, with a touch of RHS 29 D at the base of the petals.

Sepal:
Quantity: Typically 4 sepals fused at the base (stem).
Shape: The shape of sepal is lineal lanceolate.
Length: The length is about 9 mm.
Width: The width is about 2 mm.
Apex: The shape of the apex is acute.
Margin: The shape of the margin is entire.
Texture: The texture of the sepals is glabrous and shinning.
Color when opening:
   Upper surface: The upper surface of the sepal is yellow-RHS 144 A.
   Lower surface: The lower surface of the sepal is yellow-green, RHS 144 A.
Color when fully open:
   Upper surface: The upper surface of the sepal is yellow-green, RHS 144 A.
   Lower surface: The lower surface the sepal is yellow-green, RHS 144 A.

Peduncle:
Length: Every flower has a peduncle with a length of 4 mm.
Color: The peduncle is yellow-green, RHS 146 A.
Texture: The peduncle is glabrous and shinning.

REPRODUCTIVE ORGANS:

Stamen:
Number: The flower has between 4 and 7 stamens.
Color: The color of the stamens is greyed-orange, RHS 177B.

Anthers:
Number: The number of anthers is between 4 and 7.
Size: The anthers are less than 1 mm in length.
Color: The color of the anthers are yellow-orange, RHS 14 B.

Filament Color: The color of the filament is yellow-green, RHS 145 C.

Pollen Color: The color of the pollen is yellow-orange, RHS 17 C.

Pollen Amount: A medium amount of pollen with a medium pollen fertility of about 60% depending on time of year.

Pistil:
Number: The number of pistils is four.

Stigma:
Shape: The shape of stigma is round.
Color: The color of the stigma is yellow-green, RHS 145 A.

Style:
Shape: The shape of the style is thin and cylindrical.
Color: The color of style is yellow-green, RHS 144 A.

Ovary:
Color: The color of ovary is yellow-green, RHS 144 B.

Seeds:
Number: Between 50 and 60 potential seeds per ovary.
Width: The width of the seeds is less than 0.5 mm.
Length: The length of the seeds is less than 1 mm.
Shape: The shape of seeds is ellipsoidal.
Color: The color of the potential seeds is yellow-green, RHS 145 C.

Fruit (Ovary):
Shape: The shape of the fruit is cylindrical.
Width: The width of the fruit is about 1 mm.
Length: The length of the fruit is about 6 mm.
Color: The color of the fruit is yellow-green, RHS 145 A.

DISEASE RESISTANCE/SUSCEPTIBILITY: No information on disease resistance or susceptibility is currently available.

The invention has been described with reference to various specific and preferred embodiments and techniques. However, it should be understood that many variations and modifications may be made while remaining within the spirit and scope of the invention. The relevant portions of the references cited herein are incorporated by reference.

What is claimed is:

1. A Kalanchoe interspecific hybrid plant having one or more double-type flowers with at least 5 full or partial petals per flower.

2. The double-type Kalanchoe interspecific hybrid plant according to claim 1 selected from the group consisting of K. aromatica×K. blossfeldiana, K. grandiflora×K. blossfeldiana, K. laciniata×K. blossfeldiana, K. pumila×K. blossfeldiana, K. marmorata×K. blossfeldiana, K. porphyrocalux×K. blossfeldiana, K. jongmansii×K. blossfeldiana, K. pinnata×K. blossfeldiana, and K. daigremontiana×K. blossfeldiana.

3. A double-type K. laciniata×K. blossfeldiana plant having one or more double-type flowers with at least 5 full or partial petals per flower.

4. The Kalanchoe interspecific hybrid plant according to claim 1, wherein substantially all the flowers produced by said plant are double-type with at least 5 full or partial petal per flower.

5. The Kalanchoe interspecific hybrid according to claim 1, wherein a double-type flower has at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more than 40 petals per flower.

6. A method for breeding a double-type Kalanchoe interspecific hybrid plant that produces one or more flowers with at least 5 full or partial petals per flower comprising the steps of:
a. crossing a double-type K. blossfeldiana double-type plant, either as the male or female parent, with a single-type Kalanchoe plant selected from the group consisting of K. aromatica, K. grandiflora, K. laciniata, K. pumila, K. marmorata, K. porphyrocalux, K. jongmansii, K. pinnata and K. daigremontiana;
b. selecting progeny that produce one or more flowers with at least 5 full or partial petals per flower.

7. The method according to claim 6, wherein the double-type interspecific progeny plant is back-crossed with one of the parents.

8. A method for breeding a double-type Kalanchoe interspecific hybrid plant that produces one or more flowers with at least 5 full or partial petals per flower comprising the step of selfing a double-type Kalanchoe interspecific hybrid selected from the group consisting of K. aromatica×K. blossfeldiana, K. grandiflora×K. blossfeldiana, K. laciniata×K. blossfeldiana, K. pumila×K. blossfeldiana, K. marmorata×K. blossfeldiana, K. porphyrocalux×K. blossfeldiana, K. jongmansii×K. blossfeldiana, K. pinnata×K. blossfeldiana and K. daigremontiana×K. blossfeldiana.

9. A method for breeding a double-type Kalanchoe interspecific hybrid plant that produces one or more flowers with at least 5 full or partial petals per flower comprising the steps of:
a. crossing a double-type Kalanchoe interspecific hybrid selected from the group consisting of K. aromatica×K. blossfeldiana, K. grandiflora×K. blossfeldiana, K. laciniata×K. blossfeldiana, K. pumila×K. blossfeldiana, K. marmorata×K. blossfeldiana, K. porphyrocalux×K. blossfeldiana, K. jongmansii×K. blossfeldiana, K. pinnata×K. blossfeldiana and K. daigremontiana×K. blossfeldiana, as the male or female parent, with single or double-type Kalanchoe plant of the same or different species; and
b. selecting a progeny plant that produces double-type flowers.
